(19) United States
(12) Patent Application Publication
(10) Pub. No.: US 2010/0105763 A1
(43) Pub. Date: Apr. 29, 2010

(54) PHARMACEUTICAL COMPOSITION, HEALTH FOOD COMPOSITION AND INOS INHIBITORS, CONTAINING THEOPEDERIN DERIVATIVES

(75) Inventors: Heonjoong Kang, Gyeonggi-do (KR); Sang-Jip Nam, Gyeonggi-do (KR); Hyun-Sil Ko, Seoul (KR)

Correspondence Address: CANTOR COLBURN, LLP, 20 Church Street, 22nd Floor, Hartford, CT, 06103, US

(73) Assignee: SEOUL NATIONAL UNIVERSITY INDUSTRY FOUNDATION, Seoul (KR)

(21) Appl. No.: 12/519,313

(22) PCT Filed: Dec. 14, 2007
(86) PCT No.: PCT/KR07/06566

§ 371 Date: Jun. 15, 2009

(30) Foreign Application Priority Data
Dec. 15, 2006 (KR) 10-2006-0128459

(51) Int. Cl. A61K 31/357; C07D 493/04

(52) U.S. Cl. 514/452; 549/364

(57) ABSTRACT

The present invention relates to a pharmaceutical composition and a health food composition for the prevention and treatment of immune- and autoimmune diseases such as septic shock, hemorrhagic shock, rheumatoid arthritis, osteoarthritis, inflammatory bowel disease and multiple sclerosis, and metabolic diseases such as arteriosclerosis and type II diabetes, and an inducible nitric oxide synthase (iNOS) inhibitor composition containing theopederin derivatives as an active ingredient. The composition of the present invention can be effective in the prevention and treatment of immune diseases and metabolic diseases by inhibiting the excessive generation of NO by inhibiting the activity of iNOS.

### TECHNICAL FIELD

The present invention relates to a pharmaceutical composition and a health food composition for the prevention and treatment of immune- and autoimmune diseases such as septic shock, hemorrhagic shock, rheumatoid arthritis, osteoarthritis, inflammatory bowel disease and multiple sclerosis, and metabolic diseases such as arteriosclerosis and type II diabetes, and an inducible nitric oxide synthase (iNOS) inhibitor composition containing theopederin derivatives as an active ingredient. Herein, the theopederin derivatives are marine natural substances isolated and purified from Porifera.

### BACKGROUND ART

Nitrogen monoxide (NO) is known as an endothelium derived relaxing factor essential for arterio-atony which is released from vascular endothelial cells by such a stimulus as acetylcholine and acts as a factor relaxing adjacent smooth muscle cells. NO is generated from L-arginine by three kinds of nitric oxide synthases (NOS), which are eNOS (endothelial NOS), nNOS (neuronal NOS) and iNOS (inducible NOS). Unlike NO generated by eNOS or nNOS, playing important roles in nervous system, cardiovascular system and immune system, excessive production of NO by iNOS is known to induce non-specific tissue damage and insulin resistance (Nature Medicine, 2001, 7, 1138). Therefore, the specific iNOS inhibitor can be developed as a therapeutic agent for the treatment of immune diseases and metabolic diseases.

Theopederin derivatives were isolated from Theonella sp., the Porifera (J. Org. Chem. 1992, 57, 3828; J. Am. Chem. Soc. 1988, 110, 4851; J. Nat. Prod. 1993, 56, 976; Tetrahedron 1992, 48, 8369; J. Nat. Prod. 2000, 63, 704; J. Nat. Prod. 2002, 65, 59; J. Org. Chem. 1990, 55, 223; Tetrahedron 1999, 55, 13697). According to the previous reports, theopederin derivatives have cytotoxicity against P-388 murine leukemia cells (J. Org. Chem. 1992, 57, 3828) and human lung adenocarcinoma A-549 cells (J. Nat. Prod. 2002, 65, 59); antiviral effect (J. Org. Chem. 1990, 55, 223) and antifungal effect against Saccharomyces cerevisiae (Tetrahedron 1999, 55, 13697). However, no physiological activity of inhibiting iNOS activity has been reported, yet.

### DISCLOSURE

### Technical Problem

It is an object of the present invention to provide a pharmaceutical composition containing theopederin derivatives as an active ingradient for the prevention and treatment of immune- and autoimmune diseases such as septic shock, hemorrhagic shock, rheumatoid arthritis, osteoarthritis, inflammatory bowel disease and multiple sclerosis, and metabolic diseases such as arteriosclerosis and type II diabetes.

It is another object of the present invention to provide a pharmaceutical composition and a health food composition for inhibiting the inducible nitric oxide synthase (iNOS) activity.

### Technical Solution

The present invention relates to a pharmaceutical composition for the prevention and treatment of immune- and autoimmune diseases such as septic shock, hemorrhagic shock, rheumatoid arthritis, osteoarthritis, inflammatory bowel disease and multiple sclerosis, and metabolic diseases such as arteriosclerosis and type II diabetes, and a health food composition, containing theopederin derivatives as an active ingredient, and further the present invention relates to a composition for inhibiting iNOS which is excessive NO producing enzyme inducing non-specific tissue damage and insulin resistance.

The theopederin derivatives of the present invention are characteristically selected from the compounds represented by the following formula 1.

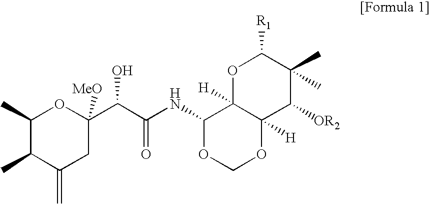

Wherein, R₁ is —CH₂OH or selected from the substituents having the following structures, and R₂ is H or C1-C5 alkyl;

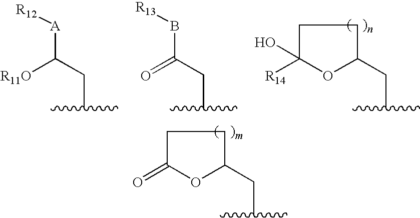

R₁₁ is H or C1-C5 alkyl, A and B are C1-C10 alkylene or alkenylene, R₁₂-R₁₄ are selected from the group consisting of —OH, —COOR₂₁ and —CONH—R₂₂, R₂₁ is H or C1-C5 alkyl, R₂₂ is a substituent having the following structure, and m and n are integers of 1-3.

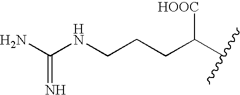

R₁ of the theopederin derivatives represented by formula 1 of the present invention is —CH₂OH or selected from the substituents having the following structures.

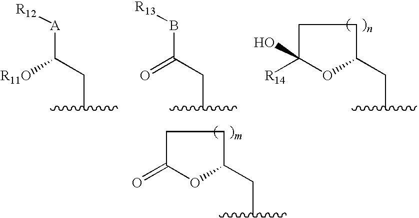

Wherein, R₁₁ is H or C1-C5 alkyl, A and B are C1-C10 alkylene or alkenylene having at least 2 double bonds, R₁₂-R₁₄ are selected from the group consisting of —OH, —COOR₂₁ and —CONH—R₂₂, R₂₁ is H or C1-C5 alkyl, R₂₂ is a substituent having the following structure, and m and n are integers of 1-3.

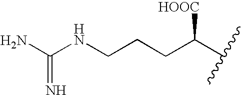

R₁₂-R₁₄ of the theopederin derivatives represented by formula 1 of the present invention are selected from the following substituents.

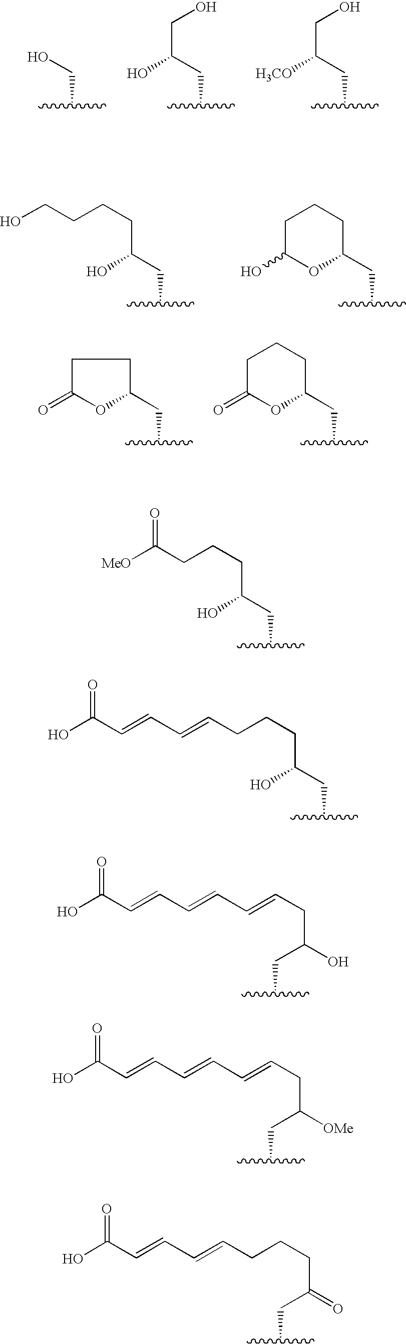

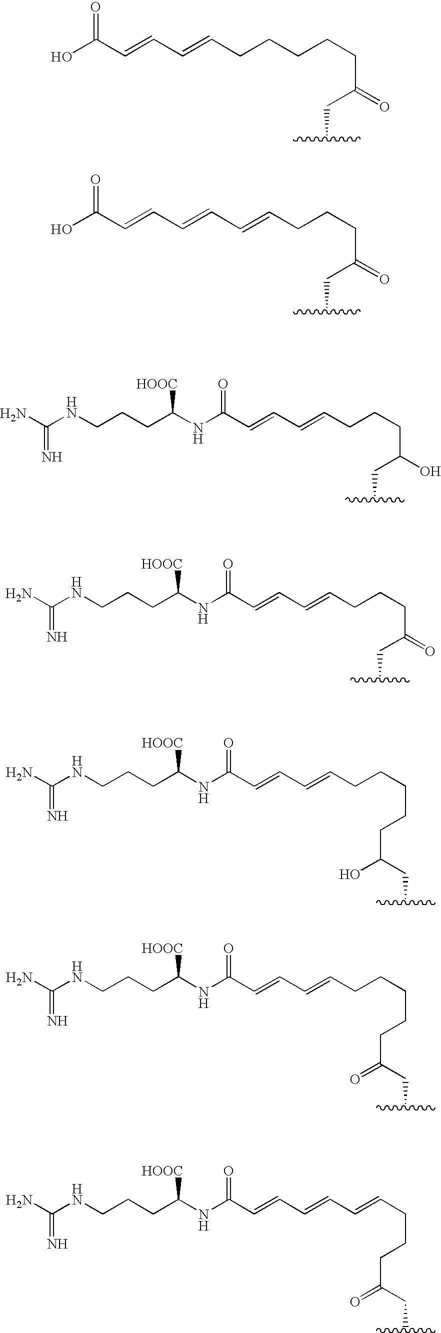

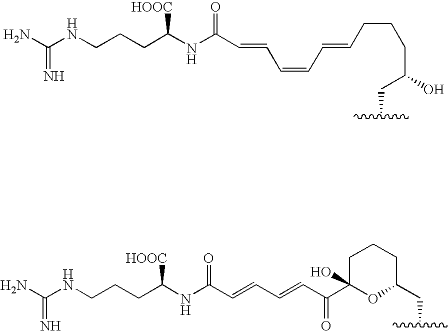

The theopederin derivatives represented by formula 1 of the invention is extracted from Porifera. The structure of the compound of formula 1 is shown in Table 1. But the structure of the compound shown in Table 1 cannot limit the spirit and scope of the present invention.

TABLE 1

| | R₁ | R₂ |
|---|---|---|
| (1) | 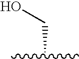 | Me |
| (2) | 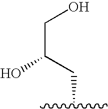 | Me |
| (3) | 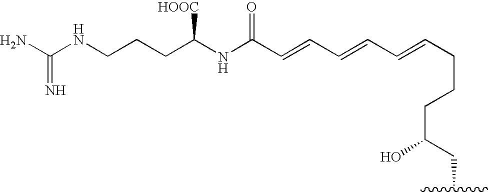 | Me |
| (4) | 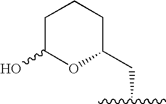 | Me |
| (5) | 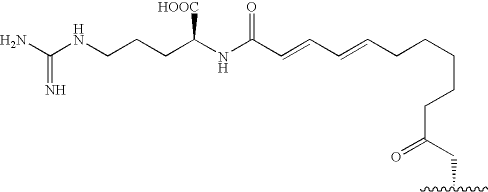 | Me |
| (6) | 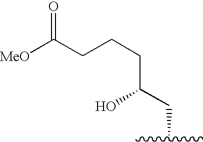 | Me |
| (7) | 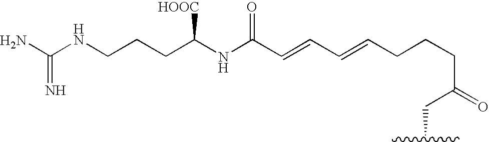 | H |
| (8) | 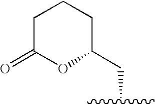 | Me |
| (9) | 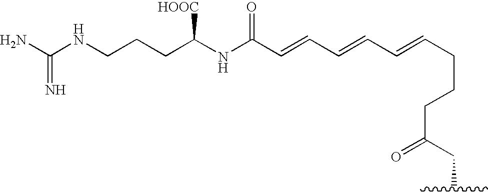 | Me |
| (10) | 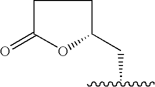 | Me |
| (11) | 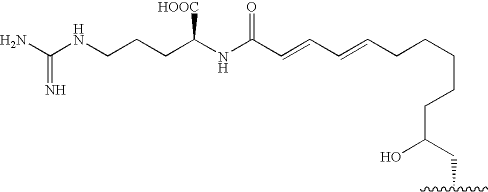 | Me |
| (12) | 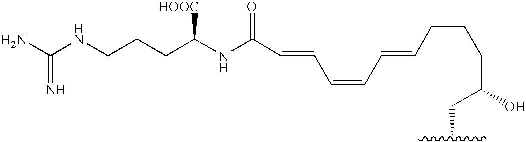 | Me |
| (13) | 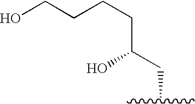 | Me |
| (14) | 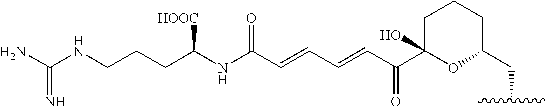 | Me |
| (15) | 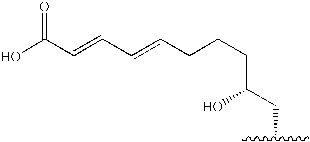 | Me |
| (16) | 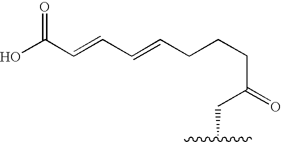 | Me |
| (17) | 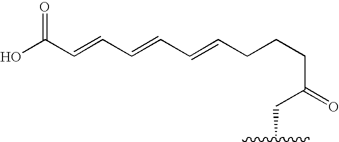 | Me |
| (18) | 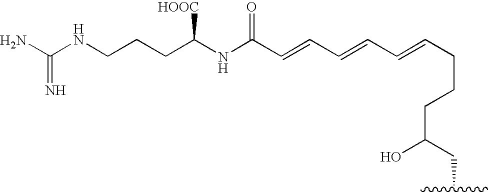 | H |
| (19) | 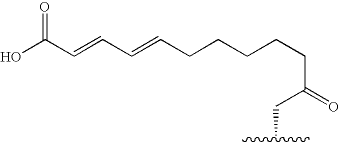 | Me |
| (20) | 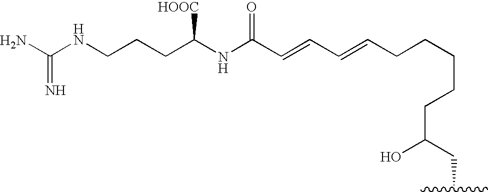 | Me |
| (21) | 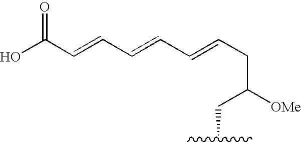 | Me |
| (22) | 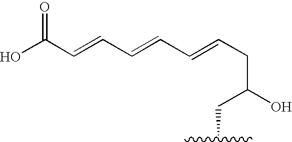 | Me |
| (23) | 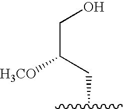 | Me |
| (24) | 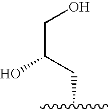 | H |

To achieve the treatment effect, the dosage of the pharmaceutical composition and the iNOS inhibitor composition of the present invention can be regulated according to the specific target compound, administration method, target subject and target disease, but generally the dosage of the active ingredient theopederin derivatives is 0.001 mg/kg-400 mg/kg. And administration frequency is once a day or preferably a few times a day. The effective dosage of the composition can be determined according to body weight, age, gender, health condition, diet, administration frequency, administration method, excretion and severity of a disease. The composition of the present invention can be administered orally or parenterally (for example, intravenous, subcutaneous and local or peritoneal injection or via visual pathway) but oral administration is preferred, for which the composition can be formulated as tablets, capsules, powders, solutions and suppositories for anal administration.

The formulations for oral administration are tablets, powders, dry syrups, chewable tablets, granules, capsules, soft capsules, pills, drinks, sublinguals, etc. In the case of powders, the content of the active ingredient can be regulated by formulations within the range of 0.01-99.9 weight %. If the content of pharmaceutically acceptable salt of the theopederin derivatives of the pharmaceutical composition of the invention exceeds the upper limit, the composition is hard to maintain its physical properties, while if the content is too small, the pharmacological effect of the active ingredient cannot be brought.

The composition of the invention formulated as tablets can be administered to a subject by any method or pathway that delivers the effective dose of the tablet with bioavailability, which can be oral administration. And the administration method or pathway can be determined according to the characteristics, stages of the target disease and other conditions. When the composition of the invention is formed as tablets, it can additionally include pharmaceutically acceptable excipients. The content and characteristics of the excipient can be determined by the solubility and chemical properties of the selected tablet, administration pathway and standard pharmaceutical practice.

Particularly, the composition of the present invention can include one or more pharmaceutically acceptable excipients as an essential ingredient along with the effective dosage of the above mentioned active ingredient. The excipient can be any solid or half-solid substance that can be a vehicle or a medium for the active ingredient and the acceptable excipients are well informed to those in the art. The excipient can be selected according to the purpose and methods of administration. For example, for the formulation of tablets, powders, chewable tablets, granules, capsules, soft capsules, pills, sublinguals or syrups, the active ingredient can be mixed with inactive non-toxic pharmaceutically acceptable excipients such as lactose and starch. The tablets of the invention can also include a binding agent such as amorphous cellulose, gum tragacanth or gelatin; a disintegrating agent such as alginic acid; a lubricant such as magnesium stearate; a glidant such as colloidal silicon dioxide; a sweetening agent such as sucrose or saccharin; a coloring agent such as peppermint or methyl salicylate; or a flavor.

The tablet can be the most preferred unit formulation for oral administration owing to its easiness of administration. If necessary, the tablet can be coated with saccharide, shellac or other enteric coating substances by the conventional methods. The tablet or capsule preferably contains 1 mg-200 mg of the active ingredient.

The theopederin derivatives of the present invention can be effectively used for the health food composition for the prevention of immune- and autoimmune diseases such as septic shock, hemorrhagic shock, rheumatoid arthritis, osteoarthritis, inflammatory bowel disease and multiple sclerosis and metabolic diseases such as arteriosclerosis and type II diabetes, and this health food composition of the invention can aid the treatment of the related diseases. The health food composition containing theopederin derivatives of the present invention is also effective in the prevention of hypertension and can aid the treatment of the related diseases.

In a preferred embodiment of the present invention, as long as the theopederin derivatives are added as an essential ingredient, other additives are not specifically limited. When the composition of the present invention is used for food or beverages, the composition can be added as it is or after being mixed with other food or ingredients, according to the conventional method.

The health food composition of the present invention is preferably added to food or beverages by 0.01-15 weight % for the total weight of the food or beverages, for example it is preferably added by 0.001-10 g and more preferably 0.01-1 g to 100 ml of the health beverages. The health beverages containing the composition of the present invention can additionally include various flavors or natural carbohydrates, etc, like other beverages. The natural carbohydrates above can be one of monosaccharides such as glucose and fructose, disaccharides such as maltose and sucrose, polysaccharides such as dextrin and cyclodextrin, and sugar alcohols such as xilytole sorbitol and erythritol.

Besides, natural sweetening agents (thaumatin, stevia extract, for example rebaudioside A, glycyrrhizin, etc.) and synthetic sweetening agents (saccharin, aspartame, etc.) can be included as a sweetening agent. The content of the natural carbohydrate is preferably 0.01-20 g and more preferably 0.1-12 g in 100 ml of the composition. In addition to the ingredients mentioned above, the composition of the present invention can include in variety of nutrients, vitamins, minerals (electrolytes), flavors including natural flavors and synthetic flavors, coloring agents and extenders (cheese, chocolate, etc.), pectic acid and its salts, alginic acid and its salts, organic acid, protective colloidal viscosifiers, pH regulators, stabilizers, antiseptics, glycerin, alcohols, carbonators which used to be added to soda, etc. The composition of the present invention can also include natural fruit juice, fruit beverages and/or fruit flesh addable to vegetable beverages. All the mentioned ingredients can be added singly or together. The mixing ratio of those ingredients does not matter in fact, but in general, each can be added by 0-20 weight part per 100 weight part of the composition of the invention.

### BEST MODE

Hereinafter, the embodiments of the present invention will be described in detail with reference to accompanying drawings.

### Example 1

### Isolation and Purification of Theopederin Derivatives (1)-(24)

The theopederin derivatives of the invention were extracted from Porifera. Extraction was performed by using methanol and dichloromethane, followed by evaporation under reduced pressure. The residue proceeded to solvent partitioning by using methanol and normal-hexane and the resultant methanol layer was dried. The residue proceeded to solvent partitioning again using ethylacetate and water and the resultant ethylacetate layer was dried. The ethylacetate extract was subjected into chromatography to give the theopederin derivatives (1)-(24) shown in Table 1.

As an alternative method, the following is used: As soon as taken from the ocean, Porifera was frozen right away. The Porifera was thawed (20 kg, wet weight) and cut into 2×2 cm sections. The first extraction was performed with the samples using 1 L of the mixed organic solvent of methanol and dichloromethane (CH₂Cl₂, 1:1 three times at room temperature. The extract was volatilized under reduced pressure to give a residue. The residue was partition-extracted by using chloroform and water.

The chloroform extract was dried under reduced pressure to give a residue. The residue proceeded to silica gel column chromatography. At that time, hexane and ethylacetate were used as eluents. 20 fractions were obtained from the column chromatography. These 20 fractions proceeded to C-18 reversed phase semi-preparative HPLC column (Polar RP C-18, 10 μm, 250×10 mm, UV detector=210 nm, flow rate: 2.5 ml/min) using acetonitrile and water as eluting solutions to give eluates. The eluates were volatilized under reduced pressure to give the amorphous theopederin derivatives (1)-(24).

### Example 2

### Identification of Chemical Structures of the Theopederin Derivatives (1)-(24)

The structures of the pure compounds (1)-(24) in Example 1 were determined on the basis of LRMS, HRMS, ¹H-NMR (nuclear magnetic resonance), ¹³C-NMR, and two-dimensional NMRs.

The compounds (1)-(24) were confirmed to be theopederin derivatives which had been already reported as marine natural substances (J. Org. Chem. 1992, 57, 3828; J. Am. Chem. Soc. 1988, 110, 4851; J. Nat. Prod. 1993, 56, 976; Tetrahedron 1992, 48, 8369; J. Nat. Prod. 2000, 63, 704; J. Nat. Prod. 2002, 65, 59; J. Org. Chem. 1990, 55, 223; Tetrahedron 1999, 55, 13697).

Compound (1) ¹H NMR (500 MHz, CDCl₃) δ 7.40 (d, 1H, J=9.8 Hz), 5.84 (dd, 1H, J=9.8, 9.8 Hz), 5.13 (d, 1H, J=6.8 Hz), 4.92 (d, 1H, J=6.8 Hz), 4.86 (d, 1H, J=1.9 Hz), 4.75 (d, 1H, J=2.0 Hz), 4.20 (s, 1H), 4.20 (dd, 1H, J=10.3, 6.9 Hz), 4.04 (dq, 1H, J=6.6, 2.8 Hz), 3.86 (dd, 1H, J=9.8, 6.9 Hz), 3.64 (m, 1H), 3.59 (m, 1H), 3.57 (s, 3H), 3.56 (m, 1H), 3.44 (d, 1H, J=10.3 Hz), 3.32 (s, 3H), 2.35 (d, 1H, J=14.1 Hz), 2.27 (dq, 1H, J=7.1, 2.8 Hz), 2.24 (ddd, 1H, J=14.1, 2.0, 1.9 Hz), 1.20 (d, 1H, J=6.6 Hz), 1.09 (s, 3H), 1.02 (d, 1H, J=7.1 Hz), 0.87 (s, 3H). ¹³C NMR (125 MHz, CdCl₃) δ 170.0, 145.1, 111.0, 100.0, 86.7, 80.2, 79.4, 74.2, 71.2, 70.6, 69.5, 61.7, 61.3, 48.5, 41.2, 39.9, 33.0, 23.3, 17.9, 14.6, 12.1, 5.84

Compound (2) ¹H NMR (500 MHz, MeOH-d₄) δ 5.80 (dd, 1H, J=9.8, 9.8 Hz), 5.20 (d, 1H, J=6.8 Hz), 4.80 (d, 1H, J=6.8 Hz), 4.80 (d, 1H, J=1.9 Hz), 4.64 (d, 1H, J=2.0 Hz), 4.28 (s, 1H), 4.16 (dd, 1H, J=10.3, 6.9 Hz), 3.88 (m, 1H), 3.88 (dd, 1H, J=9.8, 6.9 Hz), 3.64 (m, 1H), 3.56 (s, 3H), 3.44 (d, 1H, J=10.3 Hz), 3.34 (s, 3H), 2.41 (ddd, 1H, J=14.1, 2.0, 1.9 Hz), 2.31 (d, 1H, J=14.1 Hz), 2.20 (dq, 1H, J=7.1, 2.8 Hz), 1.67 (dd, 1H, J=14.1, 1.9 Hz), 1.63 (d, 1H, J=14.1 Hz), 1.00 (s, 3H), 1.28 (d, 1H, J=6.6 Hz), 0.96 (d, 1H, J=7.1 Hz), 0.86 (s, 3H)

Compound (3) ¹H (500 MHz, MeOH-d₄) δ 7.13 (dd, 1H, J=15.0, 11.2 Hz), 6.50 (dd, 1H, J=14.8, 10.7 Hz), 6.23 (dd, 1H, J=14.7, 11.3 Hz), 6.19 (dd, 1H, J=15.3, 10.7 Hz), 6.07 (d, 1H, J=15.0 Hz), 5.93 (dt, 1H, J=15.2, 6.9 Hz), 5.79 (d, 1H, J=9.3 Hz), 5.48 (d, 1H, J=6.9 Hz), 4.80 (d, 1H, J=6.9 Hz), 4.79 (br s, 1H), 4.63 (br s, 1H), 4.36 (dd, 1H, J=7.9, 5.3 Hz), 4.23 (s, 1H), 4.16 (dd, 1H, J=9.7, 6.5 Hz), 3.98 (dd, 1H, J=9.3, 6.5 Hz), 3.87 (qd, 1H, J=6.5, 2.4 Hz), 3.64 (m, 1H), 3.62 (d, 1H, J=9.6 Hz), 3.55 (s, 3H), 3.47 (dd, 1H, J=8.1, 3.6 Hz), 3.22 (s, 3H), 3.19 (m, 2H), 2.40 (br d, 1H, J=14.4 Hz), 2.32 (br d, 1H, J=14.4 Hz), 2.21 (m, 1H), 2.18 (m 1H), 2.13 (m, 1H), 1.89 (m, 1H), 1.75 (m, 1H), 1.63 (m, 2H), 1.59 (m, 1H), 1.53 (m, 2H), 1.49 (m, 1H), 1.40 (m, 1H), 1.28 (m, 1H), 1.17 (d, 3H, J=6.5 Hz), 1.00 (s, 3H), 0.96 (d, 3H, J=6.9 Hz), 0.85 (s, 3H)

Compound (4) ¹H NMR (500 MHz, CDCl₃) δ 7.52 (d, 1H, J=9.8 Hz), 5.80 (dd, 1H, J=9.8, 9.8 Hz), 5.12 (d, 1H, J=6.8 Hz), 4.86 (d, 1H, J=1.9 Hz), 4.84 (d, 1H, J=6.8 Hz), 4.74 (t, 1H, J=2.0 Hz), 4.56 (dd, 1H, J=9.6, 2.0 Hz), 4.26 (s, 1H), 4.19 (dd, 1H, J=10.3, 6.9 Hz), 4.03 (dq, 1H, J=6.6, 2.8 Hz), 3.80 (br, 1H), 3.78 (dd, 1H, J=9.8, 6.9 Hz), 3.53 (s, 3H), 3.40 (d, 1H, J=10.3 Hz), 3.40 (m, 1H), 3.33 (m, 1H), 3.30 (s, 3H), 3.18 (br, 1H), 2.36 (d, 1H, J=14.1 Hz), 2.26 (ddd, 1H, J=14.1, 2.0, 1.9 Hz), 2.25 (dq, 1H, J=7.1, 2.8 Hz), 1.80 (m, 1H), 1.80 (m, 1H), 1.64 (m, 1H), 1.61 (m, 1H), 1.53 (m, 1H), 1.41 (m, 1H), 1.22 (m, 1H), 1.19 (d, 3H, J=6.6 Hz), 1.02 (d, 3H, J=7.1 Hz), 0.98 (s, 3H), 0.95 (m, 1H), 0.84 (s, 3H)

Compound (5) ¹H NMR (500 MHz, MeOH-d₄) δ 7.10 (dd, 1H, J=15.3, 10.7 Hz), 6.20 (dd, 1H, J=14.7, 10.7 Hz), 6.08 (dt, 1H, J=14.7, 6.4 Hz), 6.01 (d, 1H, J=15.3 Hz), 5.74 (d, 1H, J=8.3 Hz), 5.16 (d, 1H, J=6.8 Hz), 4.87 (d, 1H, J=6.8 Hz), 4.79 (s, 1H), 4.63 (s, 1H), 4.37 (m, 1H), 4.29 (s, 1H), 4.14 (dd, 1H, J=9.1, 6.1 Hz), 3.96 (dd, 1H, J=8.5, 3.2 Hz), 3.88 (m, 1H), 3.88 (m, 1H), 3.63 (m, 1H), 3.55 (s, 3H), 3.24 (s, 3H), 3.21 (m, 1H), 2.48 (m, 1H), 2.38 (d, 1H, J=14.6 Hz), 2.36 (m, 1H), 2.28 (d, 1H, J=14.6 Hz), 2.18 (m, 1H), 2.18 (m, 1H), 1.89 (m, 1H), 1.73 (m, 1H), 1.62 (m, 1H), 1.62 (m, 1H), 1.52 (m, 1H), 1.43 (m, 1H), 1.18 (d, 3H, J=6.4 Hz), 1.03 (s, 3H), 0.95 (d, 3H, J=6.8 Hz), 0.86 (s, 3H)

Compound (6) ¹H NMR (500 MHz, CDCl₃) δ 7.46 (d, 1H, J=9.7 Hz), 5.89 (dd, 1H, J=9.7, 9.7 Hz), 5.15 (d, 1H, J=6.9 Hz), 4.88 (d, 1H, J=6.9 Hz), 4.85 (br s, 1H), 4.75 (br s, 1H), 4.30 (d, 1H, J=2.3 Hz), 4.23 (dd, 1H, J=10.3, 6.7 Hz), 4.01 (dq, 1H, J=6.6, 2.8 Hz), 3.93 (d, 1H, J=2.3 Hz), 3.86 (dd, 1H, J=9.7, 6.7 Hz), 3.66 (s, 3H), 3.65 (dd, 1H, J=9.3, 2.6 Hz), 3.65 (ddt, 1H, J=9.3, 7.6, 2.6 Hz), 3.57 (s, 3H), 3.47 (d, 1H, J=10.3 Hz), 3.31 (s, 3H), 2.41 (d, 1H, J=14.4 Hz), 2.38 (br d, 1H, J=14.4 Hz), 2.32 (t, 1H, J=7.5 Hz), 2.25 (dq, 1H, J=7.1, 2.8 Hz), 1.73 (dtt, 1H, J=14.1, 7.6, 7.5 Hz), 1.63 (dtt, 1H, J=14.1, 7.6, 7.5 Hz), 1.54 (dt, 1H, J=14.7, 2.6 Hz), 1.46 (dt, 1H, J=14.7, 9.3 Hz), 1.40 (m, 1H), 1.40 (m, 1H), 1.21 (d, 3H, J=Hz), 1.01 (d, 3H, J=7.1 Hz), 0.98 (s, 3H), 0.88 (s, 3H)

Compound (7) ¹H NMR (500 MHz, MeOH-d₄) δ 7.09 (dd, 1H, J=15.2, 10.8 Hz), 6.21 (dd, 1H, J=15.0, 10.7 Hz), 6.04 (ddd, 1H, J=15.0, 7.0, 7.0 Hz), 6.02 (d, 1H, J=15.2 Hz), 5.74 (dd, 1H, J=9.1, 6.0 Hz), 5.16 (d, 1H, J=6.9 Hz), 4.79 (d, 1H, J=6.9 Hz), 4.79 (dd, 1H, J=2.2, 2.2 Hz), 4.63 (dd, 1H, J=2.2, 2.2 Hz), 4.36 (dd, 1H, J=7.5, 5.3 Hz), 4.28 (s, 1H), 4.14 (dd, 1H, J=9.1, 6.0 Hz), 3.95 (dd, 1H, J=9.3, 3.0 Hz), 3.90 (dq, 1H, J=6.6, 2.6 Hz), 3.87 (dd, 1H, J=8.5, 6.0 Hz), 3.59 (br d, 1H, J=9.1), 3.55 (s, 3H), 3.24 (s, 3H), 3.23 (ddd, 1H, J=13.6, 7.0, 7.0 Hz), 3.18 (ddd, 1H, J=13.6, 7.2, 7.2 Hz), 2.53 (dd, 1H, J=16.0, 9.3 Hz), 2.47 (dd, 1H, J=16.0, 3.0 Hz), 2.47 (ddd, 1H, J=17.6, 7.4, 7.4 Hz), 2.40 (ddd, 1H, J=17.6, 7.2, 7.2 Hz), 2.37 (ddd, 1H, J=14.3, 2.2, 2.2 Hz), 2.28 (d, 1H, J=14.3 Hz), 2.19 (dq, 1H, J=7.0, 2.6 Hz), 2.14 (br q, 2H, J=7.5 Hz), 1.89 (m, 1H), 1.73 (m, 1H), 1.64 (m, 2H), 1.62 (m, 2H), 1.18 (d, 3H, J=6.6 Hz), 1.02 (s, 3H), 0.94 (d, 3H, J=7.0 Hz), 0.86 (s, 3H)

Compound (8) ¹H NMR (500 MHz, CDCl₃) δ 7.52 (d, 1H, J=9.5 Hz), 5.77 (dd, 1H, J=9.5, 9.5 Hz), 5.10 (d, 1H, J=7.1 Hz), 4.86 (br s, 1H), 4.85 (d, 1H, J=7.1 Hz), 4.73 (br s, 1H), 4.24 (d, 1H, J=2.9 Hz), 4.22 (m, 1H), 4.19 (dd, 1H, J=9.4, 6.6 Hz), 4.03 (dq, 1H, J=6.6, 2.8 Hz), 3.96 (d, 1H, J=2.9 Hz), 3.78 (dd, 1H, J=9.5, 6.6 Hz), 3.54 (s, 3H), 3.40 (d, 1H, J=9.4 Hz), 3.40 (d, 1H, J=9.4 Hz), 3.30 (s, 3H), 2.48 (ddd, 1H, J=17.5, 7.3, 5.9 Hz), 2.40 (ddt, 1H, J=17.5, 7.8, 0.9 Hz), 2.32 (d, 1H J=13.8 Hz), 2.25 (dq, 1H, J=6.5, 2.8 Hz), 2.17 (br d, 1H, J=13.8 Hz), 2.04 (m, 1H), 1.88 (m, 1H), 1.86 (m, 1H), 1.78 (m, 1H), 1.59 (m, 1H), 1.35 (m, 1H), 1.19 (d, 3H, J=6.6 Hz), 1.00 (s, 3H), 0.99 (d, 3H, J=6.5 Hz), 0.86 (s, 3H)

Compound (9) ¹H NMR (500 MHz, MeOH-d₄) δ 7.14 (dd, 1H, J=11.3 Hz), 6.52 (dt, 1H, J=14.9, 10.4 Hz), 6.28 (dd, 1H, J=14.9, 11.3 Hz), 6.18 (dd, 1H, J=15.1, 10.4 Hz), 6.08 (d, 1H, J=14.8 Hz), 5.88 (dt, 1H, J=15.1, 7.0 Hz), 5.74 (d, 1H, J=8.8 Hz), 5.16 (d, 1H, J=7.3 Hz), 4.87 (d, 1H, J=7.3 Hz), 4.79 (s, 1H), 4.63 (s, 1H), 4.36 (dd, 1H, J=7.5, 5.2 Hz), 4.28 (s, 1H), 4.14 (dd, 1H, J=9.3, 5.9 Hz), 3.95 (dd, 1H, J=8.8, 3.4 Hz), 3.89 (m, 1H), 3.87 (m, 1H), 3.60 (m, 1H), 3.54 (s, 3H), 3.24 (s, 3H), 3.21 (m, 1H), 2.49 (m, 1H), 2.44 (m, 1H), 2.38 (d, 1H, J=14.1 Hz), 2.28 (d, 1H, J=14.1 Hz), 2.20 (dd, 1H, J=6.8, 2.4 Hz), 2.18 (dd, 1H, J=14.6, 7.0 Hz), 1.90 (m, 1H), 1.74 (m, 1H), 1.62 (m, 1H), 1.62 (m, 1H), 1.60 (m, 1H), 1.19 (d, 3H, J=6.8 Hz), 1.02 (s, 3H), 0.95 (d, 3H, J=6.8 Hz), 0.86 (s, 3H)

Compound (10) ¹H NMR (500 MHz, CDCl₃) δ 7.52 (d, 1H, J=9.4 Hz), 5.81 (dd, 1H, J=9.4, 9.4 Hz), 5.13 (d, 1H, J=7.0 Hz), 4.87 (d, 1H, J=7.0 Hz), 4.86 (br s, 1H), 4.74 (br s, 1H), 4.45 (ddd, 1H, J=14.2, 8.4, 6.0 Hz), 4.27 (d, 1H, J=3.1 Hz), 4.21 (dd, 1H, J=10.2, 6.4 Hz), 4.08 (d, 1h, J=3.1 Hz), 4.03 (dq, 1H, J=6.5, 2.7 Hz), 3.82 (dd, 1H, J=9.4, 6.4 Hz), 3.56 (s, 3H), 3.44 (d, 1H, J=10.2 Hz), 3.42 (d, 1H, J=10.2 Hz), 3.30 (s, 3H), 2.51 (ddd, 1H, J=17.6, 10.0, 3.8 Hz), 2.45 (dd, 1H, J=17.6, 11.1 Hz), 2.40 (m, 1H), 2.35 (d, 1H, J=14.0 Hz), 2.26 (dq, 1H, J=7.1, 2.7 Hz), 2.21 (br d, 1H, J=14.0 Hz), 1.94 (m, 1H), 1.75 (m, 1H), 1.59 (dd, 1H, J=14.2, 8.3 Hz), 1.20 (d, 3H, J=6.5 Hz), 1.01 (d, 3H, J=7.1 Hz), 1.02 (s, 3H), 0.88 (s, 3H)

Compound (11) ¹H NMR (500 MHz, MeOH-d₄) δ 7.10 (dd, 1H, J=15.1, 10.8 Hz), 6.20 (dd, 1H, J=15.1, 10.8 Hz), 6.08 (ddd, 1H, J=15.1, 7.1, 7.1 Hz), 6.00 (d, 1H, J=15.1 Hz), 5.80 (d, 1H, J=9.3 Hz), 5.20 (d, 1H, J=6.9 Hz), 4.80 (d, 1H, J=6.9 Hz), 4.80 (dd, 1H, J=2.1, 2.1 Hz), 4.64 (dd, 1H, J=2.1, 2.1 Hz), 4.36 (dd, 1H, J=7.6, 5.2 Hz), 4.24 (s, 1H), 4.16 (dd, 1H, J=8.1, 6.4 Hz), 3.97 (dd, 1H, J=9.3, 6.4 Hz), 3.88 (dq, 1H, J=6.6, 2.5 Hz), 3.64 (d, 1H, J=8.1 Hz), 3.62 (m, 1H), 3.55 (s, 3H), 3.49 (d, 1H, J=6.7 Hz), 3.24 (s, 3H), 3.22 (ddd, 1H, J=13.5, 6.8, 6.8 Hz), 3.18 (ddd, 1H, J=13.5, 7.2, 7.2 Hz), 2.40 (ddd, 1H, J=14.3, 2.1, 2.1 Hz), 2.32 (d, 1H, J=14.3 Hz), 2.19 (dq, 1H, J=7.1, 2.5 Hz), 2.18 (q, 2H, J=7.1 Hz), 1.89 (m, 1H), 1.73 (m, 1H), 1.62 (quint, 2H, J=7.1 Hz), 1.53 (br t, 2H, J=5.9 Hz), 1.45 (m, 4H), 1.38 (m, 1H), 1.30 (m, 3H), 1.17 (d, 3H, J=6.6 Hz), 1.00 (s, 3H), 0.96 (d, 3H, J=7.1 Hz), 0.86 (s, 3H)

Compound (12) ¹H NMR (500 MHz, MeOH-d₄) δ 7.66 (dd, 1H, J=14.4, 11.9 Hz), 6.70 (dd, 1H, J=14.5, 11.7 Hz), 6.27 (dd, 1H, J=11.7, 10.7 Hz), 6.06 (d, 1H, J=14.4 Hz), 5.99 (dd, 1H, J=11.9, 10.7 Hz), 5.96 (dt, 1H, J=14.5, 7.3 Hz), 5.80 (d, 1H, J=9.3 Hz), 5.21 (d, 1H, J=6.8 Hz), 4.87 (d, 1H, J=6.8 Hz), 4.79 (s, 1H), 4.63 (s, 1H), 4.39 (dd, 1H, J=7.3, 5.4 Hz), 4.24 (s, 1H), 4.16 (dd, 1H, J=9.8, 6.3 Hz), 3.98 (dd, 1H, J=9.3, 6.3 Hz), 3.85 (dd, 1H, J=6.4, 2.4 Hz), 3.67 (m, 1H), 3.64 (m, 1H), 3.56 (s, 3H), 3.48 (dd, 1H, J=8.3, 3.9 Hz), 3.24 (m, 1H), 3.22 (s, 3H), 2.39 (d, 1H, J=14.7 Hz), 2.31 (d, 1H, J=14.7 Hz), 2.18 (m, 1H), 2.18 (m, 1H), 1.90 (m, 1H), 1.75 (m, 1H), 1.62 (m, 1H), 1.60 (m, 1H), 1.53 (m, 1H), 1.45 (m, 1H), 1.45 (m, 1H), 1.30 (m, 1H), 1.16 (d, 3H, J=6.4 Hz), 1.00 (s, 3H), 0.95 (d, 3H, J=7.3 Hz), 0.86 (s, 3H)

Compound (13) ¹H NMR (500 MHz, CDCl₃) δ 7.48 (d, 1H, J=9.6 Hz), 5.89 (t, 1H, J=10.2 Hz), 5.14 (d, 1H, J=6.6 Hz), 4.86 (d, 1H, J=6.6 Hz), 4.83 (s, 1H), 4.72 (s, 1H), 4.30 (s, 1H), 4.22 (dd, 1H, J=10.2, 6.6 Hz), 3.98 (dd, 1H, J=6.6, 2.4 Hz), 3.85 (dd, 1H, J=10.2, 6.6 Hz), 3.63 (m, 1H), 3.62 (m, 2H), 3.62 (m, 1H), 3.55 (s, 3H), 3.46 (d, 1H, J=10.2 Hz), 3.29 (s, 3H), 2.39 (d, 1H, J=13.8 Hz), 2.35 (d, 1H, J=13.8 Hz), 2.23 (dq, 1H, J=6.6, 2.4 Hz), 1.56 (m, 2H), 1.51 (dt, 1H, J=14.4, 2.0 Hz), 1.46 (m, 1H), 1.42 (m, 1H), 1.38 (m, 3H), 1.18 (d, 3H, J=6.6 Hz), 0.98 (d, 3H, J=6.6 Hz), 0.96 (s, 3H), 0.86 (s, 3H)

Compound (14) ¹H NMR (500 MHz, MeOH-d₄) δ 7.38 (dd, 1H, J=15.3, 11.4 Hz), 7.26 (dd, 1H, J=15.0, 11.4 Hz), 7.00 (d, 1H, J=15.3 Hz), 6.54 (d, 1H, J=15.0 Hz), 5.78 (d, 1H, J=9.2 Hz), 5.20 (d, 1H, J=7.0 Hz), 4.82 (dd, 1H, J=2.1, 2.1 Hz), 4.80 (d, 1H, J=7.0 Hz), 4.65 (dd, 1H, J=2.1, 2.1 Hz), 4.37 (dd, 1H, J=7.5, 5.3 Hz), 4.28 (s, 1H), 4.15 (dd, 1H, J=9.8, 7.5 Hz), 4.04 (dddd, 1H, J=11.7, 9.2, 3.5, 2.1 Hz), 3.95 (dd, 1H, J=9.2, 7.5 Hz), 3.88 (dq, 1H, J=6.6, 2.4 Hz), 3.63 (br d, 1H, J=9.8 Hz), 3.55 (s, 3H), 3.45 (dd, 1H, J=10.0, 1.8 Hz), 3.25 (s, 3H), 3.20 (m, 2H), 2.41 (ddd, 1H, J=14.3, 2.1, 2.1 Hz), 2.34 (d, 1H, J=14.3 Hz), 2.19 (dq, 1H, J=7.0, 2.4 Hz), 1.90 (m, 1H), 1.88 (m, 1H), 1.76 (m, 1H), 1.74 (m, 3H), 1.63 (m, 2H), 1.60 (m, 2H), 1.54 (ddd, 1H, J=14.0, 9.2, 1.8 Hz), 1.17 (d, 3H, J=6.6 Hz), 1.15 (m, 1H), 1.00 (s, 3H), 0.97 (d, 3H, J=7.0 Hz), 0.85 (s, 3H)

Compound (15) ¹H NMR (500 MHz, MeOH-d₄) δ 7.25 (dd, 1H, J=15.0, 10.2 Hz), 6.29 (dd, 1H, J=15.6, 10.8 Hz), 6.20 (dt, 1H, J=15.6, 8.4 Hz), 5.80 (d, 1H, J=15.0 Hz), 5.80 (d, 1H, J=9.6 Hz), 5.22 (d, 1H, J=7.2 Hz), 4.78 (d, 1H, J=7.2 Hz), 4.68 (br s, 2H), 4.17 (dd, 1H, J=10.2, 7.2 Hz), 4.16 (m, 1H), 3.97 (dd, 1H, J=9.6, 7.2 Hz), 3.94 (s, 1H), 3.67 (d, 1H, J=10.2 Hz), 3.63 (m, 1H), 3.56 (s, 3H), 3.43 (t, 1H, J=6.6 Hz), 2.72 (d, 1H, J=13.8 Hz), 2.23 (m, 1H), 2.19 (m, 1H), 2.18 (m, 1H), 2.10 (d, 1H, J=13.8 Hz), 1.58 (m, 1H), 1.50 (m, 1H), 1.50 (m, 1H), 1.48 (m, 1H), 1.44 (m, 1H), 1.28 (m, 1H), 1.07 (d, 3H, J=6.6 Hz), 1.00 (d, 3H, J=7.2 Hz), 0.99 (s, 3H), 0.85 (s, 3H)

Compound (16) ¹H NMR (500 MHz, MeOH-d₄) δ 7.04 (dd, 1H, J=15.0, 10.2 Hz), 6.18 (dd, 1H, J=15.0, 10.2 Hz), 5.98 (dt, 1H, J=15.0, 7.8 Hz), 5.82 (d, 1H, J=15.0 Hz), 5.74 (d, 1H, J=9.0 Hz), 5.20 (d, 1H, J=6.6 Hz), 4.78 (d, 1H, J=6.6 Hz), 4.69 (br s, 2H), 4.16 (m, 1H), 4.14 (m, 1H), 4.01 (s, 1H), 3.90 (m, 1H), 3.88 (m, 1H), 3.68 (d, 1H, J=10.8 Hz), 3.56 (s, 3H), 2.72 (d, 1H, J=13.8 Hz), 2.48 (m, 1H), 2.43 (m, 1H), 2.43 (m, 1H), 2.41 (m, 1H), 2.19 (dq, 1H, J=6.6, 2.0 Hz), 2.13 (m, 1H), 2.13 (m, 1H), 2.12 (d, 1H, J=13.8 Hz), 1.64 (m, 1H), 1.63 (m, 1H), 1.08 (d, 3H, J=6.6 Hz), 1.00 (s, 3H), 1.00 (d, 3H, J=6.6 Hz), 0.89 (s, 3H)

Compound (17) ¹H NMR (500 MHz, MeOH-d₄) δ 7.28 (dd, 1H, J=15.0, 11.4 Hz), 6.61 (dd, 1H, J=14.4, 10.8 Hz), 6.31 (dd, 1H, J=14.4, 11.4 Hz), 6.24 (dd, 1H, J=16.2, 7.2 Hz), 6.00 (dt, 1H, J=16.2, 7.2 Hz), 5.82 (d, 1H, J=15.0 Hz), 5.80 (d, 1H, J=9.6 Hz), 5.22 (d, 1H, J=7.2 Hz), 4.78 (d, 1H, J=7.2 Hz), 4.67 (br s, 2H), 4.16 (m, 1H), 4.16 (dd, 1H, J=10.2, 7.2 Hz), 3.97 (dd, 1H, J=9.6, 7.2 Hz), 3.95 (s, 1H), 3.67 (d, 1H, J=10.2 Hz), 3.63 (m, 1H), 3.56 (s, 3H), 3.43 (t, 1H, J=6.6 Hz), 2.72 (d, 1H, J=13.8 Hz), 2.19 (m, 1H), 2.19 (m, 1H), 2.15 (m, 1H), 2.10 (d, 1H, J=13.8 Hz), 1.57 (m, 1H), 1.51 (m, 1H), 1.51 (m, 1H), 1.47 (m, 1H), 1.42 (m, 1H), 1.28 (m, 1H), 1.07 (d, 3H, J=6.6 Hz), 1.00 (d, 3H, J=6.6 Hz), 0.99 (s, 3H), 0.85 (s, 3H)

Compound (18) ¹H NMR (500 MHz, MeOH-d₄) δ 7.14 (dd, 1H, J=15.0, 11.0 Hz), 6.52 (dd, 1H, J=14.8, 10.7 Hz), 6.26 (dd, 1H, J=14.8, 11.8 Hz), 6.21 (dd, 1H, J=14.8, 10.7 Hz), 6.06 (d, 1H, J=15.0 Hz), 5.94 (ddd, 1H, J=14.8, 7.0, 7.0 Hz), 5.81 (d, 1H, J=9.8 Hz), 5.11 (d, 1H, J=7.0 Hz), 4.81 (dd, 1H, J=2.0, 2.0 Hz), 4.73 (d, 1H, J=7.0 Hz), 4.64 (dd, 1H, J=2.0, 2.0 Hz), 4.38 (dd, 1H, J=8.1, 5.2 Hz), 4.22 (s, 1H), 4.08 (dd, 1H, J=10.5, 6.6 Hz), 4.03 (dd, 1H, J=9.8, 6.6 Hz), 4.01 (d, 1H, J=10.4 Hz), 3.87 (dq, 1H, J=6.8, 2.6 Hz), 3.65 (m, 1H), 3.45 (dd, 1H, J=8.1, 4.1 Hz), 3.23 (s, 3H), 3.20 (m, 2H), 2.39 (ddd, 1H, J=14.3, 2.0, 2.0 Hz), 2.33 (d, 1H, J=14.3 Hz), 2.20 (m, 1H), 2.19 (dq, 1H, J=6.8, 2.6 Hz), 2.14 (m, 1H), 1.90 (m, 1H), 1.74 (m, 1H), 1.63 (quint, 2H, J=7.3 Hz), 1.55 (m, 1H), 1.53 (m, 2H), 1.48 (m, 1H), 1.42 (m, 1H), 1.30 (m, 1H), 1.17 (d, 3H, J=6.8 Hz), 0.98 (s, 3H), 0.96 (d, 3H, J=6.8 Hz), 0.88 (s, 3H)

Compound (19) ¹H NMR (500 MHz, MeOH-d₄) δ 7.21 (dd, 1H, J=15.0, 10.8 Hz), 6.24 (dd, 1H, J=15.6, 10.8 Hz), 6.17 (dt, 1H, J=15.6, 8.4 Hz), 5.80 (d, 1H, J=10.2 Hz), 5.78 (d, 1H, J=15.0 Hz), 5.23 (d, 1H, J=7.2 Hz), 4.78 (d, 1H, J=7.2 Hz), 4.68 (br s, 2H), 4.17 (dd, 1H, J=10.8 Hz), 4.16 (m, 1H), 3.97 (dd, 1H, J=10.2, 7.2 Hz), 3.95 (s, 1H), 3.68 (d, 1H, J=10.8 Hz), 3.62 (m, 1H), 3.56 (s, 3H), 3.43 (dd, 1H, J=9.6, 2.0 Hz), 2.73 (d, 1H, J=13.8 Hz), 2.20 (m, 1H), 2.20 (m, 1H), 2.20 (m, 1H), 2.11 (d, 1H, J=13.8 Hz), 1.58 (m, 1H), 1.50 (m, 1H), 1.50 (m, 1H), 1.47 (m, 1H), 1.46 (m, 1H), 1.44 (m, 1H), 1.37 (m, 1H), 1.32 (m, 1H), 1.32 (m, 1H), 1.28 (m, 1H), 1.08 (d, 3H, J=6.6 Hz), 1.01 (d, 3H, J=6.6 Hz), 0.99 (s, 3H), 0.88 (s, 3H)

Compound (20) ¹H NMR (500 MHz, MeOH-d₄) δ 7.10 (dd, 1H, J=15.1, 10.8 Hz), 6.24 (dd, 1H, J=15.0, 10.8 Hz), 6.10 (ddd, 1H, J=15.0, 6.9, 6.9 Hz), 6.02 (d, 1H, J=15.1 Hz), 5.79 (d, 1H, J=9.3 Hz), 5.20 (d, 1H, J=6.9 Hz), 4.79 (d, 1H, J=6.9 Hz), 4.79 (dd, 1H, J=2.2, 2.2 Hz), 4.63 (dd, 1H, J=2.2, 2.2 Hz), 4.35 (dd, 1H, J=5.1, 7.9 Hz), 4.23 (s, 1H), 4.16 (dd, 1H, J=9.8, 6.5 Hz), 3.97 (dd, 1H, J=9.3, 6.5 Hz), 3.87 (dq, 1H, J=6.6, 2.8 Hz), 3.65 (m, 1H), 3.63 (d, 1H, J=9.8 Hz), 3.55 (s, 3H), 3.47 (dd, 1H, J=9.3, 3.1 Hz), 3.23 (s, 3H), 3.22 (ddd, 1H, J=13.6, 6.8, 6.8 Hz), 3.18 (ddd, 1H, J=13.6, 7.2, 7.2 Hz), 2.39 (ddd, 1H, J=14.3, 2.2, 2.2 Hz), 2.31 (d, 1H, J=14.3 Hz), 2.22 (m, 1H), 2.19 (dq, 1H, J=7.0, 2.8 Hz), 2.15 (m, 1H), 1.89 (m, 1H), 1.73 (m, 1H), 1.62 (quint, 2H, J=7.1 Hz), 1.57 (m, 1H), 1.53 (m, 2H), 1.49 (m, 1H), 1.43 (m, 1H), 1.29 (m, 1H), 1.17 (d, 3H, J=6.6 Hz), 1.00 (s, 3H), 0.95 (d, 3H, J=7.0 Hz), 0.85 (s, 3H)

Compound (21) ¹H NMR (500 MHz, MeOH-d₄) δ 7.04 (t, 1H, J=14.4 Hz), 6.19 (dd, 1H, J=15.0, 14.4 Hz), 6.01 (dt, 1H, J=15.0, 6.6 Hz), 5.79 (d, 1H, J=14.4 Hz), 5.67 (ddd, 1H, J=15.4, 6.5, 6.6 Hz), 5.56 (d, 1H, J=8.5 Hz), 5.10 (dd, 1H, J=15.4, 8.4 Hz), 5.04 (d, 1H, J=7.0 Hz), 4.73 (s, 1H), 4.70 (d, 1H, J=7.0 Hz), 4.56 (s, 1H), 4.18 (s, 1H), 4.06 (dd, 1H, J=9.1, 6.1 Hz), 3.82 (m, 1H), 3.81 (m, 1H), 3.50 (ddd, 1H, J=10.1, 8.4, 3.2 Hz), 3.44 (s, 3H), 3.41 (d, 1H, J=9.1 Hz), 3.22 (d, 1H, J=9.8 Hz), 3.16 (s, 3H), 3.09 (s, 3H), 2.86 (m, 1H), 2.27 (q, 1H, J=14.0 Hz), 2.13 (ddq, 1H, J=7.0, 2.3, 7.0 Hz), 1.61 (m, 1H), 1.41 (dd, 1H, J=10.6, 10.8 Hz), 1.14 (d, 3H, J=6.5 Hz), 0.90 (d, 3H, J=7.0 Hz), 0.88 (s, 3H), 0.75 (s, 3H)

Compound (22) ¹H NMR (500 MHz, MeOH-d₄) δ 7.27 (dd, 1H, J=15.3, 10.2 Hz), 6.32 (m, 1H), 6.25 (m, 1H), 5.82 (d, 1H, J=15.3 Hz), 5.72 (m, 1H), 5.68 (m, 1H), 5.40 (dd, 1H, J=15.4, 6.5 Hz), 5.18 (d, 1H, J=6.8 Hz), 4.86 (d, 1H, J=6.8 Hz), 4.75 (br s, 1H), 4.64 (br s, 1H), 4.23 (s, 1H), 4.15 (m, 1H), 4.12 (m, 1H), 3.98 (m, 1H), 3.85 (m, 1H), 3.60 (m, 1H), 3.55 (m, 1H), 3.53 (s, 3H), 3.23 (s, 3H), 2.93 (m, 1H), 2.41 (d, 1H, J=14.0 Hz), 2.32 (d, 1H, J=14.0 Hz), 2.18 (m, 1H), 1.60 (m, 1H), 1.50 (m, 1H), 1.17 (d, 3H, J=6.9 Hz), 1.00 (d, 3H, J=6.7 Hz), 0.97 (s, 1H), 0.88 (s, 3H), 0.85 (s, 1H), 0.75 (s, 3H)

Compound (23) ¹H NMR (500 MHz, CDCl₃) δ 7.54 (d, 1H, J=10.0 Hz), 5.79 (dd, 1H, J=9.7, 9.7 Hz), 5.12 (d, 1H, J=7.0 Hz), 4.85 (dd, 1H, J=2.0, 2.0 Hz), 4.84 (d, 1H, J=6.9 Hz), 4.72 (dd, 1H, J=1.9, 1.9 Hz), 4.29 (s, 1H), 4.21 (dd, 1H, J=10.4, 6.7 Hz), 4.02 (dd, 1H, J=6.6, 2.8 Hz), 3.79 (dd, 1H, J=9.7, 6.8 Hz), 3.65 (dd, 1H, J=11.9, 3.3 Hz), 3.55 (s, 3H), 3.47 (dd, 1H, J=11.9, 5.7 Hz), 3.44 (d, 1H, J=10.5 Hz), 3.41 (dd, 1H, J=8.3, 3.3 Hz), 3.29 (s, 3H), 3.24 (s, 3H), 3.20 (m, 1H), 2.36 (d, 1H, J=13.9 Hz), 2.24 (dd, 1H, J=6.9, 2.4 Hz), 2.22 (ddd, 1H, J=13.5, 2.0, 2.0 Hz), 1.55 (m, 1H), 1.55 (m, 1H), 1.20 (d, 3H, J=6.6 Hz), 1.01 (d, 3H, J=7.1 Hz), 0.97 (s, 3H), 0.85 (s, 3H)

Compound (24) ¹H NMR (500 MHz, CDCl₃) δ 7.50 (d, 1H, J=10.2 Hz), 5.87 (dd, 1H, J=10.2, 9.9 Hz), 5.08 (d, 1H, J=6.8 Hz), 4.82 (d, 1H, J=7.0 Hz), 4.80 (m, 1H), 4.80 (m, 1H), 4.31 (s, 1H), 4.14 (dd, 1H, J=10.8, 6.9 Hz), 4.00 (d, 1H, J=10.2 Hz), 3.99 (dq, 1H, J=6.7, 2.7 Hz), 3.89 (dd, 1H, J=9.6, 6.9 Hz), 3.75 (m, 1H), 3.63 (m, 3H), 3.58 (m, 1H), 3.39 (m, 1H), 2.37 (s, 1H), 3.29 (s, 3H), 2.24 (dq, 1H, J=6.9, 2.7 Hz), 1.57 (m, 3H), 1.18 (d, 3H, J=6.3 Hz), 1.01 (s, 1H), 0.99 (d, 3H, J=6.9 Hz), 0.92 (s, 3H)

### Example 3

### iNOS Inhibitory Activity Of Theopederin Derivatives (1)-(24)

Inhibition assay was performed with the compounds (1) and (2), the most representative compounds among those amorphous pure theopederin derivatives (1)-(24), to examine the iNOS inhibitory activity. Particularly, leucocyte originated animal cells (Raw264.7) were cultured in dMEM supplemented with 10% bovine serum and 1% antibiotics for 12 hours in a 5% CO₂ incubator. Lipopolysaccharide stimulating the iNOS expression and the compounds (1) and (2) were added thereto, followed by further culture for 12 hours. The amount of NO generated was investigated by measuring the color development by Griess reaction. In the meantime, iNOS inhibitor 1400W was used as a control.

The results showing the iNOS inhibitory activity of theopederin derivatives (1) and (2) are shown in Table 2. Theopederin derivatives (1) and (2) were confirmed to have excellent iNOS antagonistic activity at a very low concentration without inducing cytotoxicity.

TABLE 2
iNOS inhibitory activity and cytotoxicity of
theopederin derivatives (1) and (2)

| Compd. | iNOS (lC₅₀, nM) | Cytotoxicity (nM) |
|---|---|---|
| Compound 1 | 2.6 | 116 |
| Compound 2 | 30 | 120 |
| 1400 W | 10.0 | Not determined |

From the above results, it was confirmed that the theopederin derivatives (1)-(24) can be very effective iNOS inhibitors and be effectively used as a treatment agent for immune diseases and metabolic diseases.

### INDUSTRIAL APPLICABILITY

The theopederin derivatives of the present invention can be very effective in the prevention and treatment of immune diseases and metabolic diseases by inhibiting the excessive generation of NO by inhibiting the activity of iNOS.

1. A pharmaceutical composition for the prevention and/or treatment of immune diseases, auto-immune diseases and metabolic diseases comprising theopederin derivatives as an active ingredient.

2. The pharmaceutical composition for the prevention and/or treatment of immune diseases, auto-immune diseases and metabolic diseases according to claim 1, wherein the immune diseases and auto-immune diseases are septic shock, hemorrhagic shock, rheumatoid arthritis, osteoarthritis, inflammatory bowel disease are multiple sclerosis, and the metabolic diseases are arteriosclerosis and type II diabetes.

3. The pharmaceutical composition for the prevention and/or treatment of immune diseases, auto-immune diseases and metabolic diseases according to claim 1, wherein the theopederin derivatives are selected among the compounds represented by formula 1. 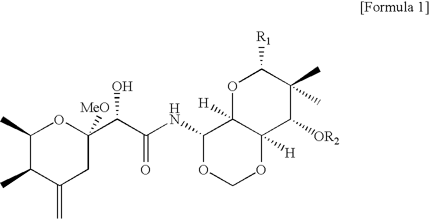
Wherein, R₁ is —CH₂OH or selected from the substituents having the following structures, and R₂ is H or C1-C5 alkyl; 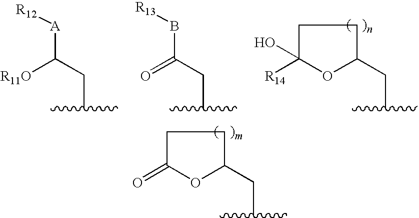
R₁₁ is H or C1-C5 alkyl, A and B are C1-C10 alkylene or alkenylene, R₁₂-R₁₄ are selected from the group consisting of —OH, —COOR₂₁ and —CONH—R₂₂, R₂₁ is H or C1-C5 alkyl, R₂₂ is a substituent having the following structure, and m and n are integers of 1-3.

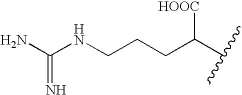

4. The pharmaceutical composition for the prevention and/or treatment of immune diseases, auto-immune diseases and metabolic diseases according to claim 3, wherein the R₁ is —CH₂OH or selected from the substituents having the following structures. 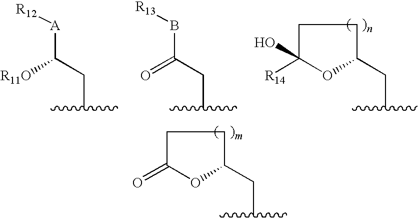
Wherein, R₁₁ is H or C1-C5 alkyl, A and B are C1-C10 alkylene or alkenylene having at least 2 double bonds, R₁₂-R₁₄ are selected from the group consisting of —OH, —COOR₂₁ and —CONH—R₂₂, R₂₁ is H or C1-C5 alkyl, R₂₂ is a substituent having the following structure, and m and n are integers of 1-3.

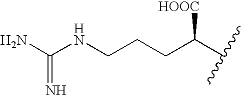

5. The pharmaceutical composition for the prevention and/or treatment of immune diseases, auto-immune diseases and metabolic diseases according to claim 4, wherein the R₁₂-R₁₄ are selected from the following substituents.

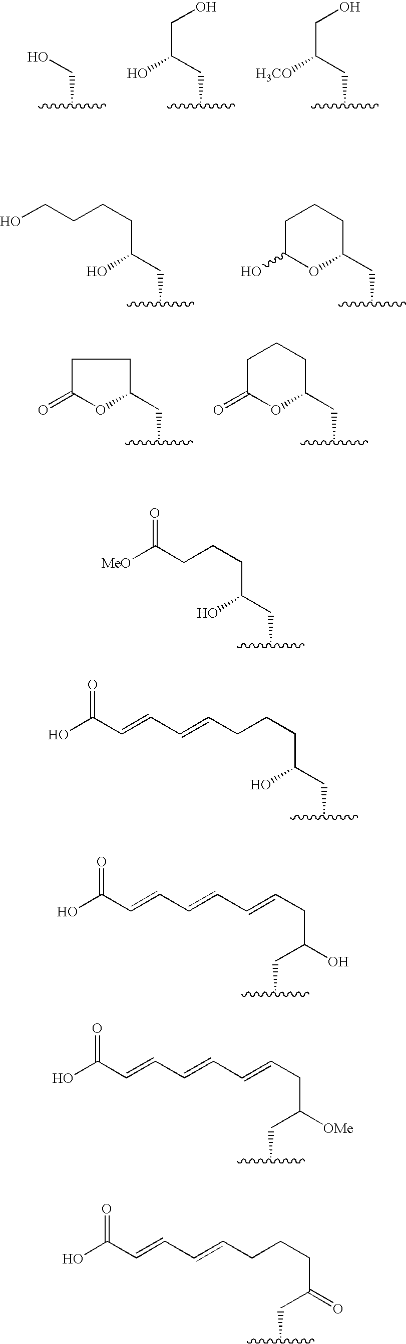

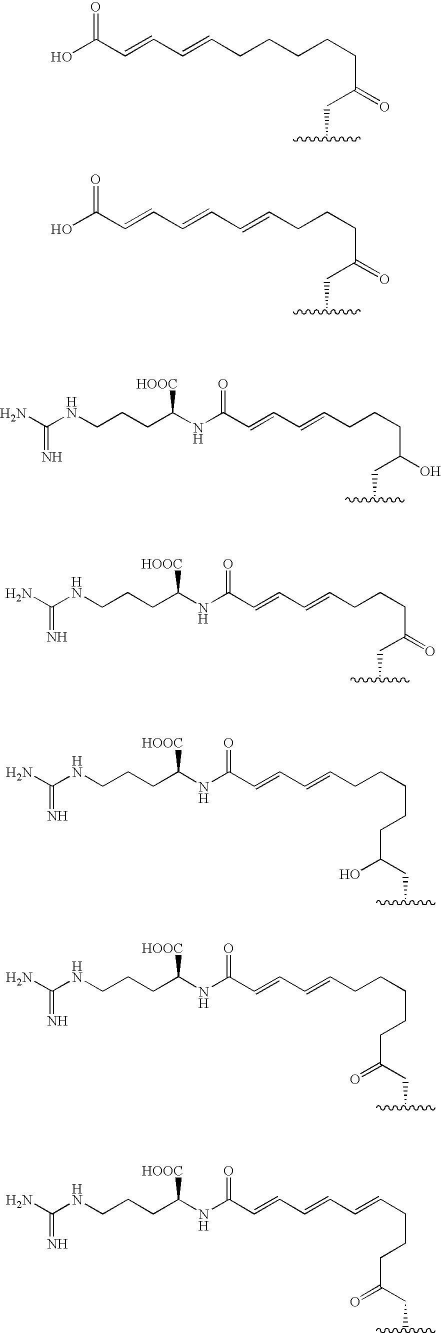

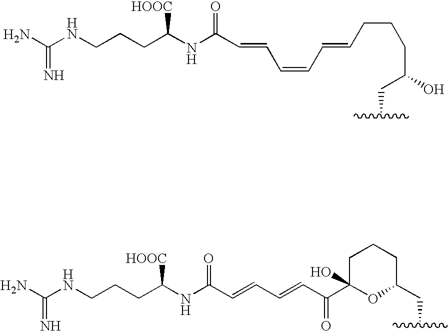

6. The pharmaceutical composition for the prevention and/or treatment of immune diseases, auto-immune diseases and metabolic diseases according to claim 1, wherein the theopederin derivatives are extracted from Porifera.

7. A pharmaceutical composition for inhibiting the inducible nitric oxide synthase (iNOS) activity comprising theopederin derivatives as an active ingredient.

8. The pharmaceutical composition for inhibiting the inducible nitric oxide synthase (iNOS) activity according to claim 7, wherein the theopederin derivatives are selected from the compounds represented by formula 1. 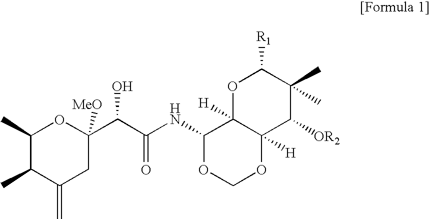
Wherein, R₁ is —CH₂OH or selected from the substituents having the following structures, and R₂ is H or C1-C5 alkyl; 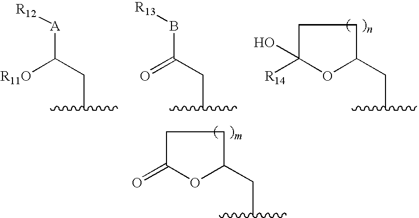
R₁₁ is H or C1-C5 alkyl, A and B are C1-C10 alkylene or alkenylene, R₁₂-R₁₄ are selected from the group consisting of —OH, —COOR₂₁ and —CONH—R₂₂, R₂₁ is H or C1-C5 alkyl, R₂₂ is a substituent having the following structure, and m and n are integers of 1-3.

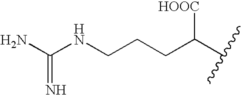

9. The pharmaceutical composition for inhibiting the inducible nitric oxide synthase (iNOS) activity according to claim 7, wherein the R₁₂-R₁₄ are selected from the following substituents.

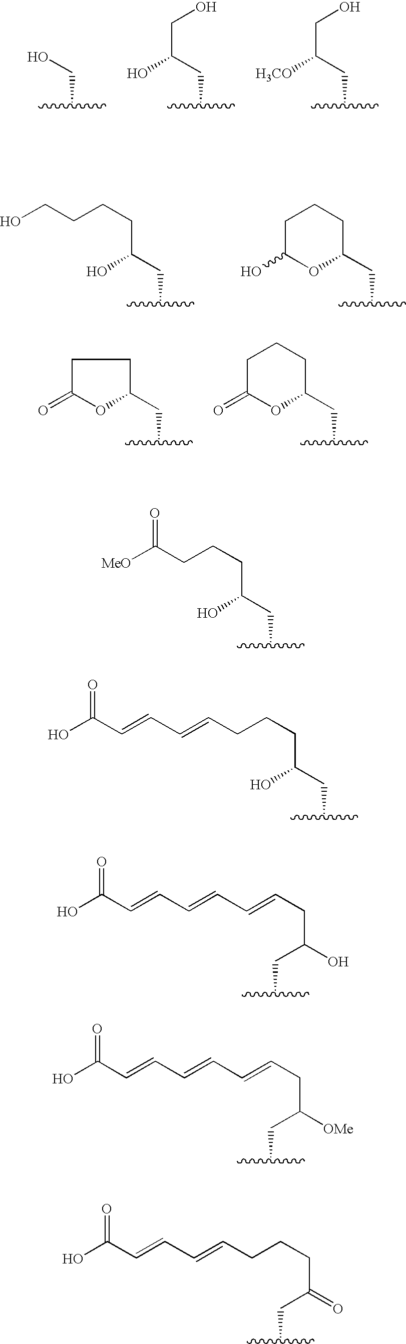

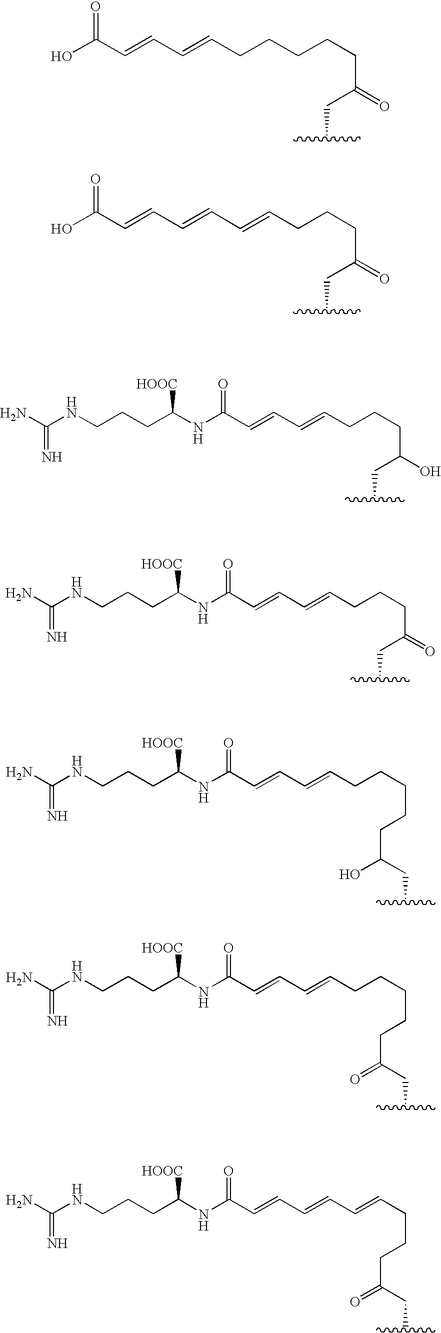

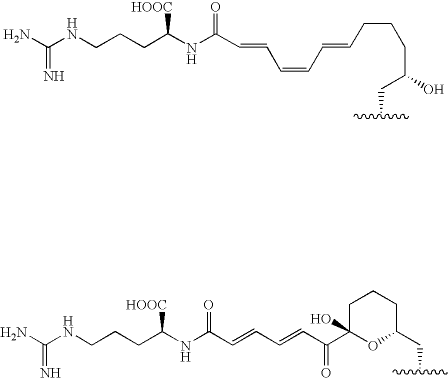

10. The pharmaceutical composition for inhibiting the inducible nitric oxide synthase (iNOS) activity according to claim 8, wherein the theopederin derivatives are extracted from Porifera.

11. A health food composition for the prevention and/or treatment of immune diseases, auto-immune diseases and metabolic diseases comprising theopederin derivatives as an active ingredient.

12. The health food composition for the prevention and/or treatment of immune diseases, auto-immune diseases and metabolic diseases according to claim 11, wherein the theopederin derivatives are selected among the compounds represented by formula 1. 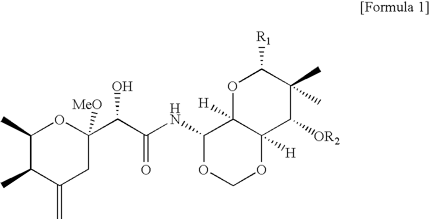
Wherein, R₁ is —CH₂OH or selected from the substituents having the following structures, and R₂ is H or C1-C5 alkyl; 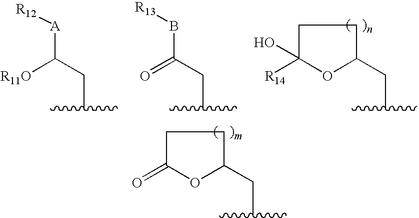
R₁₁ is H or C1-C5 alkyl, A and B are C1-C10 alkylene or alkenylene, R₁₂-R₁₄ are selected from the group consisting of —OH, —COOR₂₁ and —CONH—R₂₂, R₂₁ is H or C1-C5 alkyl, R₂₂ is a substituent having the following structure, and m and n are integers of 1-3.

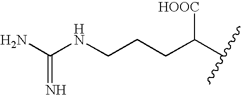

13. The health food composition for the prevention and/or treatment of immune diseases, auto-immune diseases and metabolic diseases according to claim 12, wherein the R₁₂-R₁₄ are selected from the following substituents.

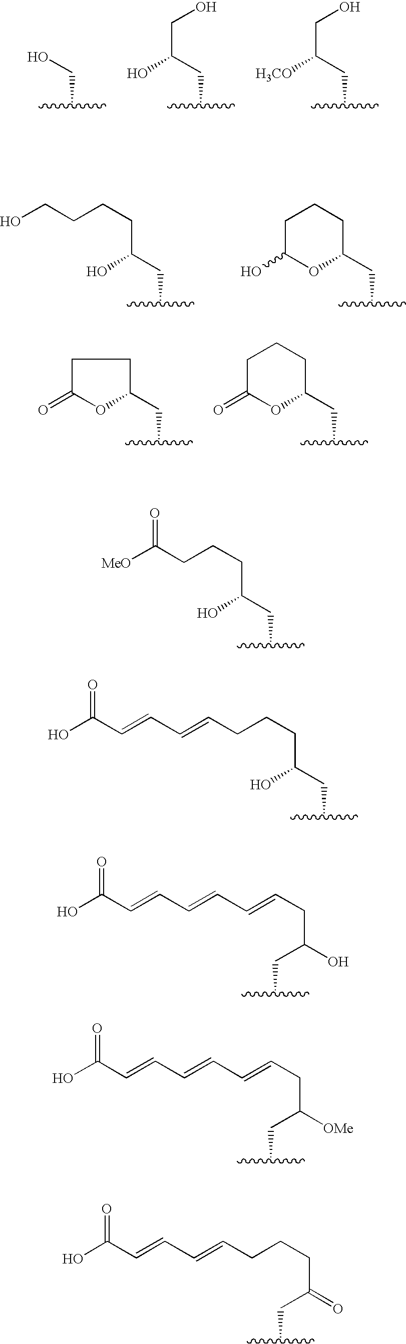

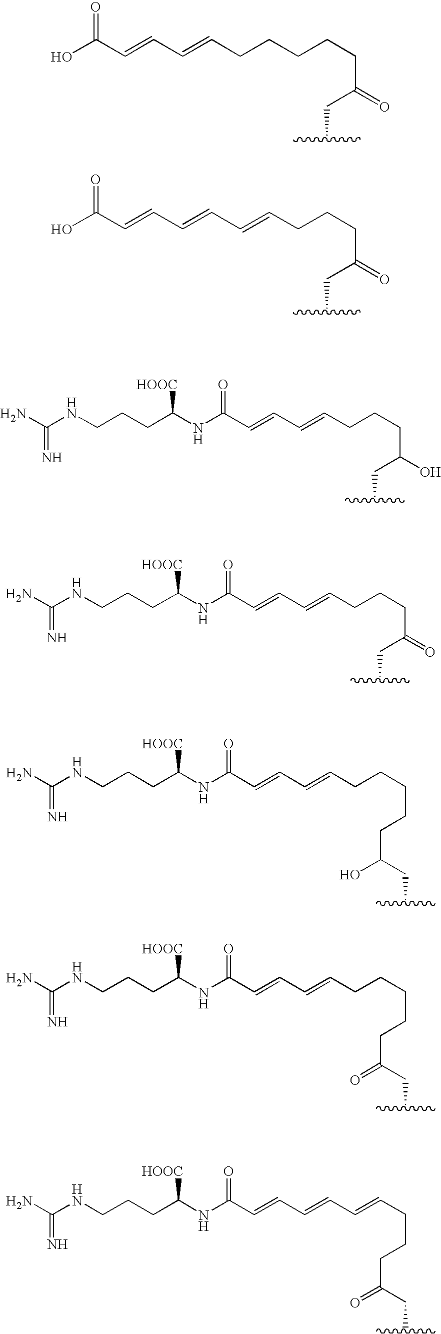

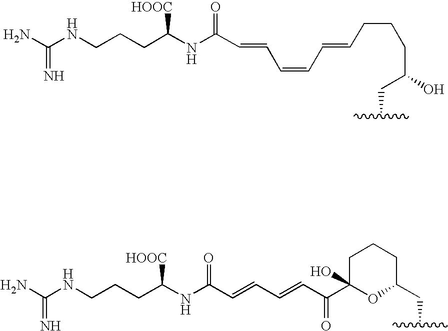

14. The health food composition for the prevention and/or treatment of immune diseases, auto-immune diseases and metabolic diseases according to claim 11, wherein the theopederin derivatives are extracted from Porifera.
